(19) United States
(12) Patent Application Publication
(10) Pub. No.: US 2023/0042232 A1
(43) Pub. Date: Feb. 9, 2023

(54) MECHANICAL METAMATERIAL-TETHERED BREATHABLE ELECTRONIC SKIN SENSOR PATCH

(71) Applicants: KOREA INSTITUTE OF SCIENCE AND TECHNOLOGY, Seoul (KR); Seoul National University R&DB Foundation, Seoul (KR)

(72) Inventors: Hyunjung YI, Seoul (KR); Wonseop HWANG, Seoul (KR); Seongjin PARK, Seoul (KR); In-Suk CHOI, Seoul (KR); Myoung-Gyu LEE, Seoul (KR); Juhee KIM, Seoul (KR)

(21) Appl. No.: 17/511,576

(22) Filed: Oct. 27, 2021

(30) Foreign Application Priority Data
Aug. 4, 2021 (KR) 10-2021-0102651

(51) Int. Cl. A61B 5/145; A61B 5/00; A61B 5/28; A61B 5/296

(52) U.S. Cl. CPC A61B 5/14517; A61B 5/683; A61B 5/28; A61B 5/296; A61B 2562/227

(57) ABSTRACT

Disclosed is an electronic skin sensor patch which is attached to a skin of a user and measures a bio signal, the electronic skin sensor patch including: a patch body including a frame which is formed with an opening and is made of a mechanical metamaterial; a sensing unit disposed on a first region of the patch body; a sensor system unit disposed on a second region of the patch body and configured to maintain a nonadherent state with the skin; and a wiring disposed along the frame of the patch body and configured to connect the sensing unit and the sensor system unit.

### CROSS-REFERENCE TO RELATED APPLICATION

This application claims priority to and the benefit of Korean Patent Application No. 10-2021-0102651 filed in the Korean Intellectual Property Office on Aug. 4, 2021, the entire contents of which are incorporated herein by reference.

### BACKGROUND OF THE INVENTION

### (a) Field of the Invention

The present disclosure relates to a skin sensor patch used while being attached to a skin of a user.

### (b) Description of the Related Art

With the advent of the Internet of Things era, where things and things or things and people are connected, the role of wearable devices is being emphasized. In line with this trend, wearable devices for measuring the interaction between the body and the external environment are being studied.

Customized technology that measures biometric information non-invasively and long-term, efficiently manages personal health and adopts the measured biometric information to treatment based on the biometric information is in the spotlight as a technology that can change the paradigm of the future medical and health care industry. Recently, in particular, research on a skin attached sensor which is attached to a skin to monitor a bio-signal is also being actively conducted. The bio-signal provides important information for biomedical devices, and multiple biosensors are essentially required to obtain individual signals from multiple points in a wide area.

However, since the human skin is composed of an open system in which water evaporation, sweat secretion, and the like continuously occur, when it is desired to obtain bio-signals for a long time by using a sensor, it is necessary to consider not only the movement of the human body, but also the transepidermal water loss in which water evaporates through the skin or sweating.

When water that needs to be continuously evaporated through the skin is not appropriately discharged due to the sensor attached to the skin, the user may feel uncomfortable due to wearing the sensor for a long time and risks, such as skin itching and skin necrosis, may also follow. Further, the adhesion of a sensor element to the human body is significantly reduced due to water that is not appropriately discharged and remains between the skin and the sensor attached to the skin, thereby causing a side effect of reducing the accuracy of the bio-signal to be measured.

Accordingly, there is a need for a skin-attached sensor capable of overcoming the limitations of the existing skin-attached sensor and capable of controlling breathability and moisture permeability to enable long-term monitoring of multiple bio-signals.

The above information disclosed in this Background section is only for enhancement of understanding of the background of the invention, and therefore it may contain information that does not form the prior art that is already known in this country to a person of ordinary skill in the art,

### SUMMARY OF THE INVENTION

The present invention has been made in an effort to provide an electronic skin sensor patch in which a sensor circuit is tethered to a mechanical metamaterial and which has breathability and stretchability.

However, the object to be solved in the exemplary embodiments of the present invention is not limited to the foregoing object, and may be variously extended in the scope of the technical spirit included in the present invention.

An exemplary embodiment of the present invention provides an electronic skin sensor patch which is attached to a skin of a user and measures a bio signal, the electronic skin sensor patch including: a patch body including a frame which is formed with an opening and is made of a mechanical metamaterial; a sensing unit disposed on a first region of the patch body; a sensor system unit disposed on a second region of the patch body and configured to maintain a nonadherent state with the skin; and a wiring disposed along the frame of the patch body and configured to connect the sensing unit and the sensor system unit.

The electronic skin sensor patch may further include a sensor system unit cover coupled to the frame of the patch body with the sensor system unit interposed therebetween in the second region of the patch body.

The sensor system unit cover may include a nonadherent surface on a surface facing the skin.

The electronic skin sensor patch may further include a wiring cover coupled to the frame of the patch body with the wiring interposed therebetween.

The sensing unit may be configured to have one exposed surface to be in contact with the skin.

The sensing unit may include an electrocardiogram (ECG) electrode or an electromyogram (EMG) electrode.

The electronic skin sensor patch may further include a spacer interposed between the ECG electrode or the EMG electrode and the frame.

The sensing unit may include a body fluid sensor configured to collect and detect a body fluid.

The body fluid sensor may include: an opening formed layer including a body fluid passage configured to discharge the body fluid; and an electrode layer positioned in the opening formed layer and configured to detect a current flowing through the body fluid collected to the body fluid passage,

The frame of the mechanical metamaterial may include a plurality of basic displacement unit bodies, and in each of the plurality of basic displacement unit bodies, m polygonal basic unit cells may be positioned while being adjacent to each other, m isolation parts may be formed between the m basic unit cells, and a junction part which connects the basic unit cells to each other may be formed between the basic unit cells, and the junction part may include a junction part pattern in which an outer junction part positioned at an outer edge of the basic unit cell and an inner junction part that is not in contact with the outer edge of the basic unit cell are sequentially repeated, and

Herein, m is an integer of 4 or 6.

The basic displacement unit body may include a first opening having a variable size, of which an initial unfolding angle is larger than 0° and is equal to or smaller than 15°, at a center.

The first opening may have a 3-pointed star shape.

The basic unit cell may include a second opening at a center.

The second opening may have a triangular shape.

The six second openings may be disposed around each first opening.

The frame made of the mechanical metamaterial may include a first frame part extending in a horizontal zigzag direction and a second frame part extending in a vertical zigzag direction, and the first frame part and the second frame part cross each other to form a crossing point, and an opening surrounded by the first frame part and the second frame part which connects the adjacent crossing points in horizontal and vertical directions may be included.

The opening may include a center opening part and a plurality of branch opening parts integrally extending in directions shifted from the center opening part.

The center opening part may have a quadrangular shape, and the branch opening part may include four branch opening parts extending from sides of the center opening part, respectively.

Each of the first frame part and the second frame part may include an angled crest and valley.

Each of the first frame part and the second frame part may include a round crest and valley.

According to the electronic skin sensor patches of the exemplary embodiments, by a metamaterial directly attached to the skin is applied for fixing the skin sensor, the electronic skin sensor patch has high breathability and mechanical properties, such as elastic modulus, similar to that of the skin, so that a stable and comfortable fit may be secured for a long time.

Further, the sensor system circuit board of the electronic skin sensor patch is not directly attached to the skin, so that when a sensor system is developed, the degree of freedom for the mechanical properties and breathability requirements of the system is high, and the present invention is based on the patch system in which the sensor and the system are integrated, so that the degrees of completion and utilization of technology are high.

Furthermore, the electronic skin sensor patch of the exemplary embodiments is a platform technology and is a highly scalable technology applicable to various types of skin sensors.

### BRIEF DESCRIPTION OF THE DRAWINGS

FIG. 1 is a diagram illustrating an example of an electronic skin sensor patch which is attached to various parts of a skin of a body and measures various bio signals.

FIG. 2 is a top plan view illustrating an electronic skin sensor patch according to an exemplary embodiment.

FIG. 3 is an exploded perspective view of the electronic skin sensor patch illustrated in FIG. 2.

FIG. 4 is a graph representing a change in effective modulus according to a tilt angle with respect to a reference line of a stretching direction of a mechanical metamaterial applied to the electronic skin sensor patch illustrated in FIG. 2.

FIG. 5 is a cross-sectional view taken along line V-V of FIG. 2.

FIG. 6 is a cross-sectional view taken along line VI-VI of FIG. 2.

FIG. 7 is a top plan view illustrating an electronic skin sensor patch according to another exemplary embodiment.

FIG. 8 is an exploded perspective view of the electronic skin sensor patch illustrated in FIG. 7.

FIG. 9 is a cross-sectional view taken along line IX-IX of FIG. 7.

FIG. 10 is a top plan view illustrating an electronic skin sensor patch according to still another exemplary embodiment.

FIG. 11 is a graph representing a change in effective modulus according to a tilt angle with respect to a reference line of a stretching direction of a mechanical metamaterial applied to the electronic skin sensor patch illustrated in FIG. 10.

FIG. 12 is a top plan view illustrating an electronic skin sensor patch according to yet another exemplary embodiment.

FIG. 13 is a graph representing a change in effective modulus according to a tilt angle with respect to a reference line of a stretching direction of a mechanical metamaterial applied to the electronic skin sensor patch illustrated in FIG. 12.

FIG. 14 is a top plan view illustrating an electronic skin sensor patch according to still yet another exemplary embodiment.

FIG. 15 is a graph representing a change in effective modulus according to a tilt angle with respect to a reference line of a stretching direction of a mechanical metamaterial applied to the electronic skin sensor patch illustrated in FIG. 14.

### DETAILED DESCRIPTION OF THE EMBODIMENTS

Hereinafter, exemplary embodiments of the present invention will be described in detail with reference to the accompanying drawings so that those skilled in the art may easily implement the present invention. The drawings and description are to be regarded as illustrative in nature and not restrictive. Like reference numerals designate like elements throughout the specification. Further, some constituent elements in the drawing may be exaggerated, omitted, or schematically illustrated, and a size of each constituent element does not reflect the actual size entirely.

Further, the accompanying drawings are provided for helping to easily understand exemplary embodiments disclosed in the present specification, and the technical spirit disclosed in the present specification is not limited by the accompanying drawings, and it will be appreciated that the present invention includes all of the modifications, equivalent matters, and substitutes included in the spirit and the technical scope of the present invention.

Terms including an ordinary number, such as first and second, are used for describing various constituent elements, but the constituent elements are not limited by the terms. The terms are used only to discriminate one constituent element from another constituent element.

Further, it will be understood that when an element such as a layer, film, region, or substrate is referred to as being “on” another element, it can be directly on the other element or intervening elements may also be present. In contrast, when an element is referred to as being “directly on” another element, there are no intervening elements present. Further, when an element is “on” a reference portion, the element is located above or below the reference portion, and it does not necessarily mean that the element is located “on” in a direction opposite to gravity.

In the present application, it will be appreciated that terms “including” and “having” are intended to designate the existence of characteristics, numbers, steps, operations, constituent elements, and components described in the specification or a combination thereof, and do not exclude a possibility of the existence or addition of one or more other characteristics, numbers, steps, operations, constituent elements, and components, or a combination thereof in advance. Accordingly, unless explicitly described to the contrary, the word “comprise” and variations such as “comprises” or “comprising”, will be understood to imply the inclusion of stated elements but not the exclusion of any other elements.

Further, in the entire specification, when it is referred to as “on a plane”, it means when a target part is viewed from above, and when it is referred to as “on a cross-section”, it means when the cross-section obtained by cutting a target part vertically is viewed from the side.

Further, throughout the specification, when it is referred to as “connected”, this does not only mean that two or more constituent elements are directly connected, but may mean that two or more constituent elements are indirectly connected through another constituent element, are physically connected, electrically connected, or are integrated even though two or more constituent elements are referred as different names depending on a location and a function.

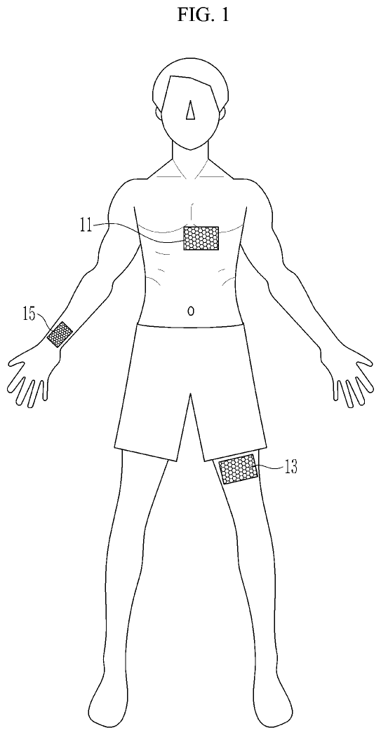

FIG. 1 is a diagram illustrating an example of an electronic skin sensor patch which is attached to various parts of a skin of a body and measures various bio signals.

Referring to FIG. 1, electronic skin sensor patches 11, 13, and 15 are attached to a skin of a user to be used for measuring bio signals. The electronic skin sensor patches 11, 13, and 15 may form adherent layers or adherent materials on a surface facing the skin and be attached to the skin of the user.

The electronic skin sensor patches 11, 13, and 15 may be configured to measure various bio signals according to configurations of a sensor system unit and a sensing unit. The electronic skin sensor patches 11, 13, and 15 may be configured to perform at least one of an electrocardiogram (ECG) measurement, an electromyogram (EMG) measurement, a pulse measurement, and a body fluid detection of the user, and may be used by being attached to different parts of the skin of the body of the user according to the measurement target.

For example, the electronic skin sensor patch 11 may be attached to a chest of the user and used for the electrocardiogram measurement and body fluid detection, the electronic skin sensor patch 13 may be attached to a leg of the user and be used for the electromyogram measurement and body fluid detection, and the electronic skin sensor patch 15 may be attached to a wrist of the user and be used for pulse measurement and body fluid detection.

As described above, the electronic skin sensor patch of the present disclosure may be used by being selectively attached to an appropriate position of the skin of the user according to the target desired to be measured or detected.

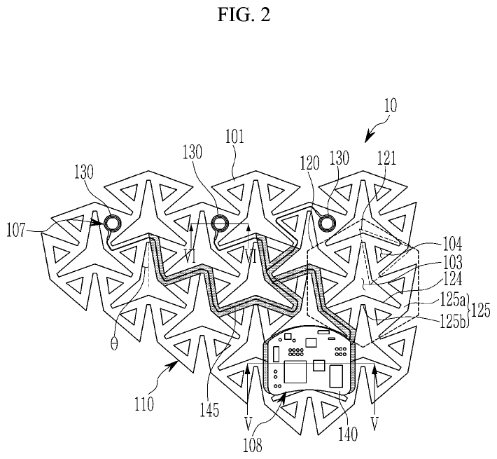

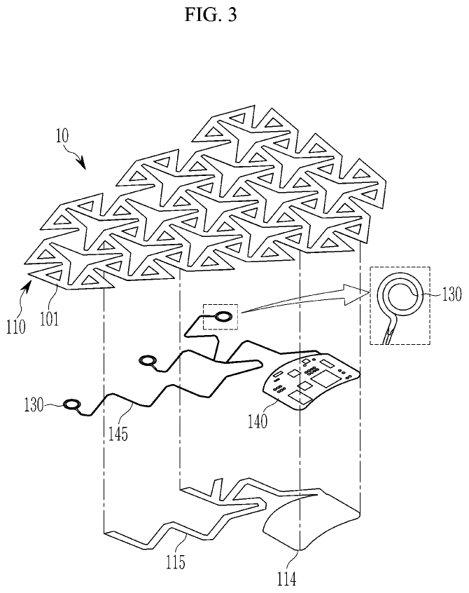

FIG. 2 is a top plan view illustrating an electronic skin sensor patch according to an exemplary embodiment, and FIG. 3 is an exploded perspective view of the electronic skin sensor patch illustrated in FIG. 2.

Referring to FIG. 2, an electronic skin sensor patch 10 according to the present exemplary embodiment may be formed of a skin sensor patch for measuring electrocardiogram (ECG). The electronic skin sensor patch 10 includes a patch body 110 including a frame 101 made of a mechanical metamaterial, and sensing units 130, a sensor system unit 140, and a wiring 145 which are fixed to the patch body 110 and measure bio signals.

The frame 101 made of the mechanical metamaterial may have an auxetic structure in which a Poisson's ratio is a negative value. The frame 101 made of the mechanical metamaterial includes a plurality of basic displacement unit bodies 120. The basic displacement unit body 120 may m polygonal basic unit cells 121 which are positioned while being adjacent to each other. m isolation parts 124 are formed between the m basic unit cells 121, and junction parts 125 which connect the basic unit cells 121 each other may be formed between the basic unit cells 121. The junction part 125 may have a junction part pattern in which outer junction parts 125a positioned at the outer edge of the basic unit cell 121 and inner junction part 125b which are not in contact with the outer edge of the basic unit cell 121 are sequentially repeated. The basic displacement unit body 120 may have the inherent form that is activated by changing relative positions of the m basic unit cells 121 according to the junction part pattern.

In the present exemplary embodiment, m is set to 6 to form the mechanical metamaterial frame 101 having a Kagome structure. Accordingly, the basic displacement unit body 120 may include six triangular basic unit cells 121. Six isolation parts 124 are formed between the six basic unit cells 121, and the junction part 125 may have the junction part pattern in which three outer junction parts 125a positioned at the outer edge of the basic unit cell 121 and three inner junction parts 125b that are not in contact with the outer edge of the basic unit cell 121 are sequentially repeated.

Accordingly, the basic displacement unit body 120 may have a first opening 103, of which an initial unfolding angle θ is larger than 0 and is equal to or smaller than 15°, in the center, and the first opening 103 has the initial unfolding angle θ that is larger than 0, so that the basic displacement unit body 120 may have a 3-pointed star shape. Herein, the initial unfolding angle θ may be defined as ½ of the angle between the adjacent basic unit cells 121. When the initial unfolding angle θ is 0, the opening is not formed, and when initial unfolding angle θ is larger than 15°, an elongation rate decreases, which is lower than 30% that is the general deformation range of the skin, so that the user may feel uncomfortable when wearing the electronic skin sensor patch. That is, the first opening 103 is unfolded so as to have a predetermined area even in an initial state, and the area of the first opening 103 may be further increased according to application of tension from the outside. Accordingly, the first opening 103 may be formed of a variable opening. The present structure configured as described above is elongated while being unfolded, and is in the already elongated state due to the initial opening, so that as the initial unfolding angle increases, the maximum structural elongation may decrease.

In this case, the triangular basic unit cell 121 may have a triangular second opening 104 therein. Accordingly, an aperture ratio of the patch body 110 is improved, thereby maximally securing breathability of the electronic skin sensor patch 10. The patch body 110 of the mechanical metamaterial frame 101 may be set to have an overall porosity of approximately 50% by combining the initial unfolding angle θ of the first opening 103 and an area of the second opening 104.

Further, the patch body 110 may be entirely asymmetrical with respect to the center line, and the vertical length of the right portion may be formed to be longer than that of the left portion. The electronic skin sensor patch 10 may be attached right under the left chest for the measurement of the electrocardiogram, and may have a planar structure in which the lower left end of the approximately rectangular shape is removed (omitted) so that the patch body 110 is not attached to the abdomen where the skin is relatively deformed a lot in the out-of-plane direction.

The sensing unit 130 may include a configuration which is in contact with the skin of the user and is capable of detecting a bio signal according to use. In the present exemplary embodiment, the sensing unit 130 may include an ECG electrode for measuring electrocardiogram. The sensing unit 130 may be disposed on a first region 107 of the patch body 110. The first region 107 of the patch body 110 does not vertically penetrate, and the upper portion of the sensing unit 130 is closed and the lower portion thereof may be exposed. Accordingly, when the electronic skin sensor patch 10 is attached to the skin, one surface of the sensing unit 130 is exposed, so that the sensing unit 130 may be in contact with the skin, but may not be exposed to the outside.

In the sensing unit 130, the three ECG electrodes may be disposed in the three basic unit cells 121, which are spaced apart from each other at uniform intervals, in the upper portion of the patch body 110, respectively. Herein, the second opening 104 may not be formed in the basic unit cells 121 in which the sensing units 130 are disposed.

The sensor system unit 140 may receive the bio signal detected from the sensing unit 130, analyze the received bio signal through necessary signal processing, and transmit a result of the analysis. The sensor system unit 140 may include various constituent elements, such as a data transmitting device and an energy device, as well as a sensor driving circuit, and may also wirelessly transmit the measured sensor data. The sensor system unit 140 may be manufactured as a sensor system based on a flexible printed circuit board (FPCB), and may be disposed on a second region 108 of the patch body 110. The second region 108 may be located in the right lower portion of the patch body 110.

The wiring 145 may connect the sensing unit 130 and the sensor system unit 140 to transmit a current and a signal between the sensing unit 130 and the sensor system unit 140. The wiring 145 may be configured to extend along the frame 101 of the patch body 110.

Referring to FIG. 3, the electronic skin sensor patch 10 according to the present exemplary embodiment may be divided into three layers. That is, the patch body 110 including the frame 101 made of the mechanical metamaterial may form a first layer, the sensing unit 130, the wiring 145, and the sensor system unit 140 may be connected with each other to form a second layer, and a sensor system unit cover 114 and a wiring cover 115 covering the sensor system unit 140 and the wiring 145 may be connected to each other to form a third layer.

The patch body 110 forming the first layer may be formed of a polymer film that is soft, breathable, and stretchable, and for example, Tegaderm™ that is a biomedical dressing film may be used. That is, the mechanical metamaterial frame 101 may be manufactured by using Tegaderm™. For another example, the patch body 110 may include nonwoven, polyurethane, and thermoplastic elastomer that are breathable medical film materials.

The sensor system unit cover 114 forming the third layer may be coupled with the patch body 110 forming the first layer with the sensor system unit 140 forming the second layer interposed therebetween in the second region 108.

In this case, the sensor system unit 140 may be configured to maintain nonadherent state on the skin of the user. To this end, the sensor system unit cover 114 may be formed with an adhesive layer on an upper surface facing the sensor system unit 140, but may include a non-adhesive surface on a surface facing the skin. Accordingly, even when the electronic skin sensor patch 10 is attached to the skin, the sensor system unit 140 may be separated from the skin and tethered to the patch body 110.

Further, the wiring cover 115 forming the third layer may be coupled with the patch body 110 forming the first layer with the wiring 145 forming the second layer interposed therebetween. Accordingly, the wiring 145 may be fixed in a buried state along the frame 101 of the patch body 110.

The sensor system unit cover 114 and the wiring cover 115 forming the third layer may also be formed of a breathable and stretchable polymer film, and for example, Tegaderm™ that is a biomedical dressing film may be used.

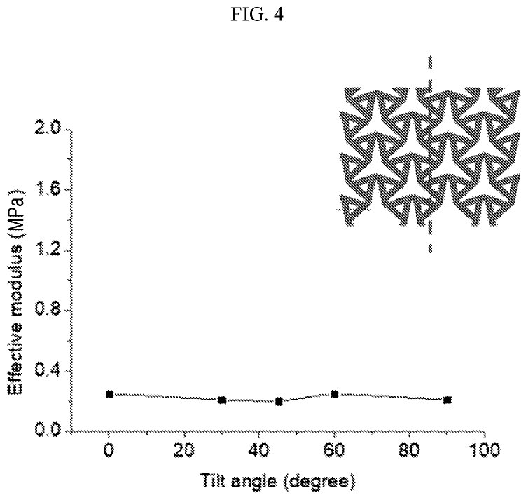

FIG. 4 is a graph representing a change in effective modulus according to a tilt angle with respect to a reference line of a stretching direction of the mechanical metamaterial applied to the electronic skin sensor patch illustrated in FIG. 2.

Referring to FIG. 4, it can be seen that when effective elastic modulus of the patch body 110 including the mechanical metamaterial frame 101 in the Kagome structure applied to the electronic skin sensor patch 10 according to the present exemplary embodiment, is calculated while the tensile direction with respect to the reference line is changed, a fairly uniform effective elastic modulus of about 0.2 MPa is maintained within the tilt angle of 0° to 90°. That is, it can be seen that the patch body 110 has an isotropic structure with little change in the elastic modulus depending on the tensile direction.

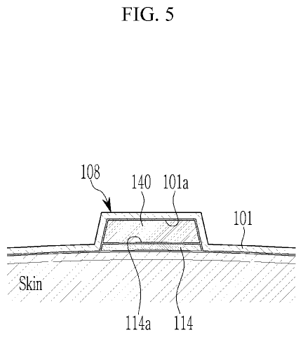

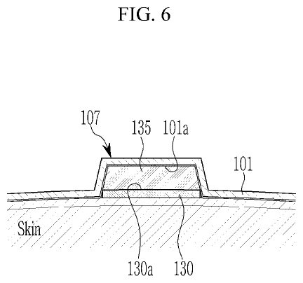

FIG. 5 is a cross-sectional view taken along line V-V of FIG. 2, and FIG. 6 is a cross-sectional view taken along line VI-VI of FIG. 2.

Referring to FIG. 5, the sensor system unit 140 may be located on a lower surface of the frame 101 in the second region 108 of the patch body 110. An adhesive layer 101a is formed on the lower surface of the frame 101, and the sensor system unit 140 may be primarily fixed to the frame 101 by the adhesive layer 101a. An adhesive used as a biomedical adhesive may be applied to the adhesive layer 101a, and for example, an Si-based adhesive or an acrylate-based adhesive may be applied.

Further, the sensor system unit cover 114 may be coupled with the frame 101 while covering the sensor system unit 140 on the lower surface of the frame 101. An adhesive layer 114a is formed on a surface facing the sensor system unit 140, so that the sensor system unit cover 114 may secondarily fix the sensor system unit 140 while being coupled with the frame 101. In this case, an adhesive layer ay not be formed on the surface facing the skin of the user in the sensor system unit cover 114. Accordingly, the second region 108 of the patch body 110 in which the sensor system unit 140 is located may tether the sensor system unit 140 while being separated from the skin.

The sensor system unit 140 may be manufactured as the sensor system based on a flexible printed circuit board (FPCB), and thus may include a circuit including a device, such as a driving IC and Bluetooth low energy (BLE).

Referring to FIG. 6, the sensing unit 130 may be located on a lower surface of the frame 101 in the first region 107 of the patch body 110. The adhesive layer 101a is formed on the lower surface of the frame 101, and the sensing unit 130 may be fixed to the frame 101 by the adhesive layer 101a. In this case, a spacer 135 may be interposed between the sensing unit 130 and the lower surface of the frame 101, so that the sensing unit 130 may be fixed to the frame 101. Accordingly, the spacer 135 is coupled to the lower surface of the frame 101, and the sensing unit 130 may be coupled to the spacer 135 to fix the sensing unit 130 and the frame 101. The spacer 135 may be formed of an elastic body, and include, for example, ecoflex, polydimethysiloxane (PDMS), or polyurethane, and may serve to maintain a thickness so that the sensing unit 130 formed of a thin ECG electrode is positioned closer to the skin of the user.

In the exemplary embodiment described with reference to FIGS. 2 to 6, the skin sensor patch for ECG measurement has been described, but the electronic skin sensor patch in the foregoing structure may be formed of a skin sensor patch for electromyogram (EMG) measurement. In this case, the sensing unit includes an EMG electrode for EMG measurement, and the shape of the patch body frame may also be changed according to the change in an attachment position for EMG measurement, which also belongs to the scope of the present invention.

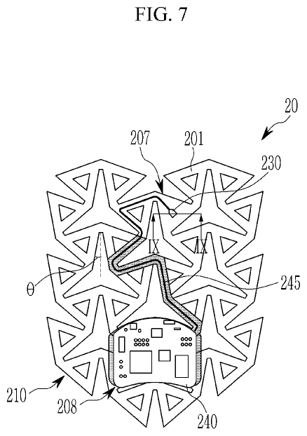

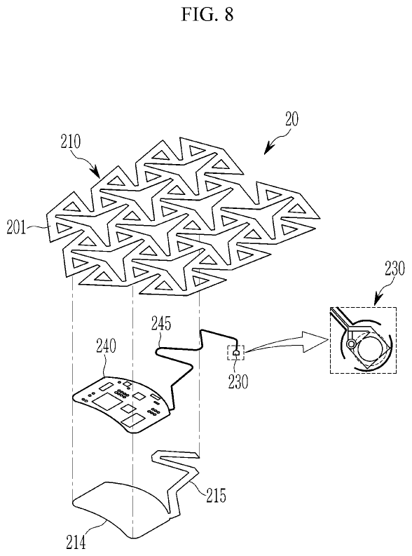

FIG. 7 is a top plan view illustrating an electronic skin sensor patch according to another exemplary embodiment, and FIG. 8 is an exploded perspective view of the electronic skin sensor patch illustrated in FIG. 7.

Referring to FIG. 7, an electronic skin sensor patch 20 according to the present exemplary embodiment may be formed of a body fluid sensor patch for collecting and analyzing a body fluid, and may be formed of, for example, a sweat sensor patch for collecting and analyzing sweat. The electronic skin sensor patch 20 includes a patch body 210 including a frame 201 made of a mechanical metamaterial, and sensing units 230, a sensor system unit 240, and a wiring 245 which are fixed to the patch body 210 and measure bio signals.

In the present exemplary embodiment, the mechanical metamaterial of the frame 201 configuring the patch body 210 may have the same structure as the structure of the frame 101 applied to the electronic skin sensor patch 10 according to the exemplary embodiment illustrated in FIG. 2. That is, the patch body 210 may be formed of the mechanical metamaterial frame 201 having the Kagome structure.

In the present exemplary embodiment, the patch body 210 may be horizontally symmetrically formed as a whole with respect to a center line. The electronic skin sensor patch 20 may be attached to various parts of the skin of the body for detecting sweat, and for example, the electronic skin sensor patch 20 may be attached to the skin of a chest, an arm, a leg, and the like to detect sweat discharged from the skin and measure and analyze a flow rate, a composition, and the like of the sweat. The patch body 210 may have a planar structure having a length longer than a width.

The sensing unit 230 may include a configuration capable of being in contact with the skin of the user to detect and monitor a body fluid, such as sweat. The sensing unit 230 may be disposed on a first region 207 of the patch body 210. In the first region 207 of the patch body 210, an upper portion of the sensing unit 230 is partially exposed, and a lower portion thereof may be exposed. Accordingly, when the electronic skin sensor patch 20 is attached to the skin, the sensing unit 230 may be configured so that one surface of the sensing unit 230 is exposed to be in contact with the skin.

The sensor system unit 240 may receive the bio signal detected from the sensing unit 230, analyze the received bio signal through necessary signal processing, and transmit a result of the analysis. The sensor system unit 240 may include various constituent elements, such as a data transmitting device and an energy device, as well as a sensor driving circuit, and may also wirelessly transmit the measured sensor data. The sensor system unit 240 may be manufactured as a sensor system based on an FPCB, and may be disposed on a second region 208 of the patch body 210. The second region 208 may be located in the lower portion of the patch body 210.

The wiring 245 may connect the sensing unit 230 and the sensor system unit 240 to transmit a current and a signal between the sensing unit 230 and the sensor system unit 240. The wiring 245 may be configured to extend along the frame 201 of the patch body 210.

Referring to FIG. 8, the electronic skin sensor patch 20 according to the present exemplary embodiment may be divided into three layers. That is, the patch body 210 including the frame 201 made of the mechanical metamaterial may form a first layer, the sensing unit 230, the wiring 245, and the sensor system unit 240 may be connected with each other to form a second layer, and a sensor system unit cover 214 and a wiring cover 215 covering the sensor system unit 240 and the wiring 245 may be connected to each other to form a third layer.

The patch body 210 forming the first layer may be formed of a polymer film that is breathable and stretchable, and may use, for example, Tegaderm™ that is a biomedical dressing film. That is, the mechanical metamaterial frame 201 may be manufactured by using Tegaderm™.

The sensor system unit cover 214 forming the third layer may be coupled with the patch body 210 forming the first layer with the sensor system unit 240 forming the second layer interposed therebetween in the second region 208. In this case, the sensor system unit 240 may be configured to maintain nonadherent state on the skin of the user. To this end, the sensor system unit cover 214 may be formed with an adhesive layer on an upper surface facing the sensor system unit 240, but may include a non-adhesive surface on a surface facing the skin. Accordingly, even when the electronic skin sensor patch 20 is attached to the skin, the sensor system unit 240 may be separated from the skin and tethered to the patch body 210.

Further, the wiring cover 215 forming the third layer may be coupled with the patch body 210 forming the first layer with the wiring 245 forming the second layer interposed therebetween. Accordingly, the wiring 245 may be fixed in a buried state along the frame 201 of the patch body 210.

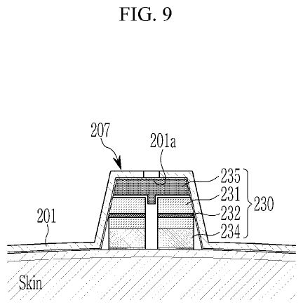

FIG. 9 is a cross-sectional view taken along line IX-IX of FIG. 7.

Referring to FIG. 9, the sensing unit 230 may be located on a lower surface of the frame 201 in the first region 207 of the patch body 210. The adhesive layer 201a is formed on the lower surface of the frame 201, and the sensing unit 230 may be fixed to the frame 201 by the adhesive layer 201a.

The sensing unit 230 may include an opening formed layer 231 forming a body fluid passage so that the body fluid is discharged in a direction away from the skin of the user. For example, when sweat is discharged through the sweat glands of the skin, the body fluid passage of the opening formed layer 231 may be used as a passage in which the discharged sweat is collected and moves.

The body fluid passage of the opening formed layer 231 may have a predetermined form which allows the discharged sweat to effectively move and be discharged in the direction away from the skin. Further, the body fluid passage may have a predetermined form capable of effectively detecting a current flowing through sweat. The body fluid passage may be formed in, for example, a cylindrical shape, or may also have a form of which a circumference decreases as the circumference is away from the skin attached surface.

The sensing unit 230 may include an electrode layer 232 for detecting the current flowing through the body fluid of the user collected through the body fluid passage. The electrode layer 232 may be located in the opening formed layer 231, and may be at least partially exposed to the body fluid passage and be in contact with the body fluid. Accordingly, the electrode layer 232 of the sensing unit 230 may detect the current flowing through the sweat while the sweat discharged through the sweat glands of the skin of the user moves through the body fluid passage. The current flowing through the sweat detected in the sensing unit 230 may be used for generating sensing data.

The sensing unit 230 may also include a hydrophobic layer 234 at a lower end of the opening formed layer 231, and include a hydrophilic layer 235 at an upper end of the opening formed layer 231. The hydrophobic layer 234 may promote an action of smoothly collecting the body fluid to the body fluid passage, and the hydrophilic layer 235 may easily and effectively promote the discharge of the body fluid moving through the body fluid passage. Further, a channel may be formed between the opening formed layer 231 and the hydrophilic layer 235, or at least a part of the frame 201 corresponding to the hydrophilic layer 235 may penetrate to promote the smooth discharge of the body fluid.

In the foregoing, one example of the sensing unit 230 which detects and monitors the body fluid, such as sweat, has been illustrated and described, but the scope of the present invention is not limited to the structure of the sensing unit 230, and various structures of the body fluid sensor which are capable of absorbing, moving, and detecting the body fluid may be applied, which also belong to the scope of the present invention.

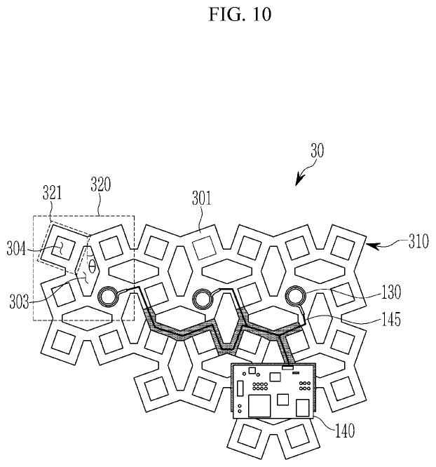

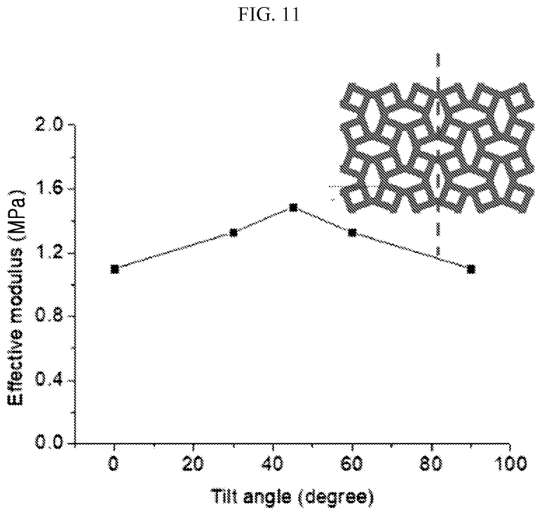

FIG. 10 is a top plan view illustrating an electronic skin sensor patch according to still another exemplary embodiment, and FIG. 11 is a graph representing a change in effective modulus according to a tilt angle with respect to a reference line of a stretching direction of a mechanical metamaterial applied to the electronic skin sensor patch illustrated in FIG. 10.

Referring to FIG. 10, an electronic skin sensor patch 30 according to the present exemplary embodiment may be formed of a skin sensor patch for measuring ECG. The electronic skin sensor patch 30 includes a patch body 310 including a frame 301 made of a mechanical metamaterial, and sensing units 130, a sensor system unit 140, and a wiring 145 which are fixed to the patch body 310 and measure bio signals.

The frame 301 of the mechanical metamaterial may have an auxetic structure in which a Poisson's ratio is a negative value. The frame 301 made of the mechanical metamaterial include a plurality of basic displacement unit bodies 320. The basic displacement unit body 320 may m polygonal basic unit cells 321 which are positioned while being adjacent to each other. M isolation parts are formed between the m basic unit cells 321, and junction parts which connect the basic unit cells 321 each other may be formed between the basic unit cells 321. The junction part may have a junction part pattern in which outer junction parts positioned at the outer edge of the basic unit cell 321 and inner junction part which are not in contact with the outer edge of the basic unit cell 321 are sequentially repeated. The basic displacement unit body 320 may have the inherent form that is activated by changing relative positions of the m basic unit cells 321 according to the junction part pattern.

In the present exemplary embodiment, m is set to 4 to form the mechanical metamaterial frame 301 having a rotating square structure. Accordingly, the basic displacement unit body 320 may include four square basic unit cells 321. Four isolation parts may be formed between the four basic unit cells 321, and the junction part may have the junction part pattern in which two outer junction parts positioned at the outer edge of the basic unit cell 321 and two inner junction part which are not in contact with the outer edge of the basic unit cell 321 are sequentially repeated.

Accordingly, the basic displacement unit body 320 may have a first opening 303, of which an initial unfolding angle θ is larger than 0 and is equal to or smaller than 15°, at the center thereof, and the first opening 303 has the initial unfolding angle θ larger than 0, so that the basic displacement unit body 320 may be formed in a rectangular or octagonal shape depending on the hinge (junction part) structure. Herein, the initial unfolding angle θ may be defined as ½ of the angle between the adjacent basic unit cells 321. When the initial unfolding angle θ is 0, the opening is not formed, and when initial unfolding angle θ is larger than 15°, an elongation rate decreases, which is lower than 30% that is the general deformation range of the skin, so that the user may feel uncomfortable when wearing the electronic skin sensor patch. That is, the first opening 303 is unfolded so as to have a predetermined area even in an initial state, and the area of the first opening 303 may be further increased according to application of tension from the outside. Accordingly, the first opening 303 may be formed of a variable opening.

In this case, the quadrangular basic unit cell 321 may have a second opening 304 shaped like a quadrangle therein. Accordingly, an aperture ratio of the patch body 310 is improved, thereby maximally securing breathability of the electronic skin sensor patch 30. The patch body 310 of the mechanical metamaterial frame 301 may be set to have an overall porosity of approximately 50% by combining the initial unfolding angle θ of the first opening 303 and an area of the second opening 304.

In the present exemplary embodiment, the sensing unit 130, the sensor system unit 140, and the wiring 145 may have the same configurations and characteristics as those of the exemplary embodiment described with reference to FIGS. 2 and 3, so that the repeated descriptions will be omitted.

Referring to FIG. 11, it can be seen that when effective elastic modulus of the patch body 310 including the mechanical metamaterial frame 301 in the rotating square structure, applied to the electronic skin sensor patch 30 according to the present exemplary embodiment, is calculated while the tensile direction with respect to the reference line is changed, a generally uniform effective elastic modulus in the range of 1.0 to 1.6 MPa is maintained within the tilt angle of 0° to 90°. That is, it can be seen that the change in the elastic modulus according to the tensile direction is not large. However, in the rotating square structure, the change in the elastic modulus according to the direction is larger than that in the Kagome structure, so that it is possible to set an appropriate patch attachment direction according to the skin tensile direction.

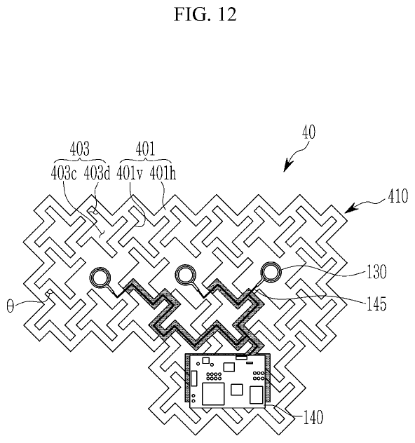

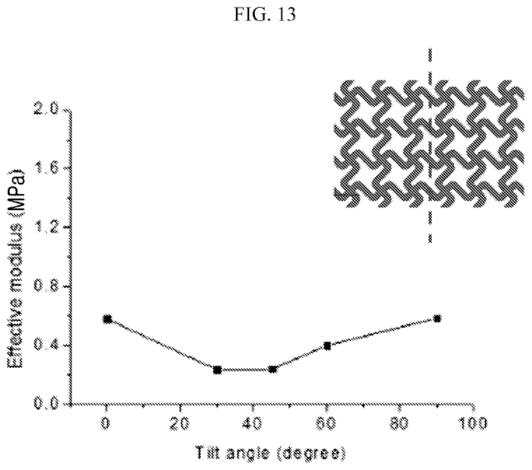

FIG. 12 is a top plan view illustrating an electronic skin sensor patch according to yet another exemplary embodiment, and FIG. 13 is a graph representing a change in effective modulus according to a tilt angle with respect to a reference line of a stretching direction of a mechanical metamaterial applied to the electronic skin sensor patch illustrated in FIG. 12.

Referring to FIG. 12, an electronic skin sensor patch 40 according to the present exemplary embodiment may be formed of a skin sensor patch for measuring ECG. The electronic skin sensor patch 40 includes a patch body 410 including a frame 401 made of a mechanical metamaterial, and sensing units 130, a sensor system unit 140, and a wiring 145 which are fixed to the patch body 410 and measures bio signals.

In the present exemplary embodiment, the mechanical metamaterial of the frame 401 configuring the patch body 410 includes a first frame part 401h extending in a horizontal zigzag direction and a second frame part 401v extending in a vertical zigzag direction, and the first frame part 401h and the second frame part 401v may cross each other to form a crossing point. In this case, an opening 403 surrounded by the first frame part 401h and the second frame part 401v, which connect the adjacent crossing points in the horizontal and vertical directions, may be formed in the patch body 401.

The opening 403 formed in the patch body 410 may include a center opening part 403c and a plurality of branch opening parts 403d integrally extending in directions shifted from the center opening part 403c. The center opening part 403c may have an approximately quadrangular shape, and in this case, the branch opening part 403d may include four branch opening parts 403d extending from the sides of the quadrangular center opening part 403c, respectively.

In the present exemplary embodiment, the first frame part 401h and the second frame part 401v may configure the mechanical metamaterial frame 401 in a Logenz structure having an angled crest and valley, respectively.

In the present exemplary embodiment, the sensing unit 130, the sensor system unit 140, and the wiring 145 may have the same configurations and characteristics as those of the exemplary embodiment described with reference to FIGS. 2 and 3, so that the repeated descriptions will be omitted.

Referring to FIG. 13, it can be seen that when effective elastic modulus of the electronic skin sensor patch 40 according to the present exemplary embodiment, is calculated while the tensile direction with respect to the reference line is changed, a generally uniform effective elastic modulus in the range of 0.2 to 0.8 MPa is maintained within the tilt angle of 0° to 90°. That is, it can be seen that the change in the elastic modulus according to the tensile direction is not large. However, in the present structure, the change in the elastic modulus according to the direction is larger than that in the Kagome structure, so that it is possible to set an appropriate patch attachment direction according to the skin tensile direction.

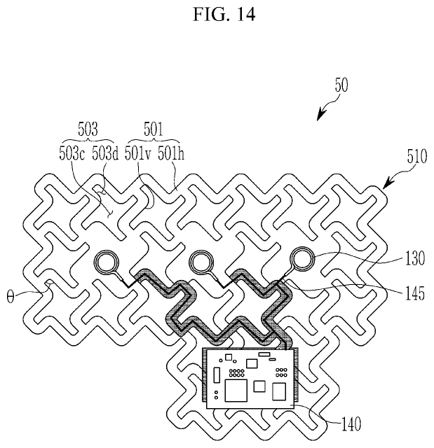

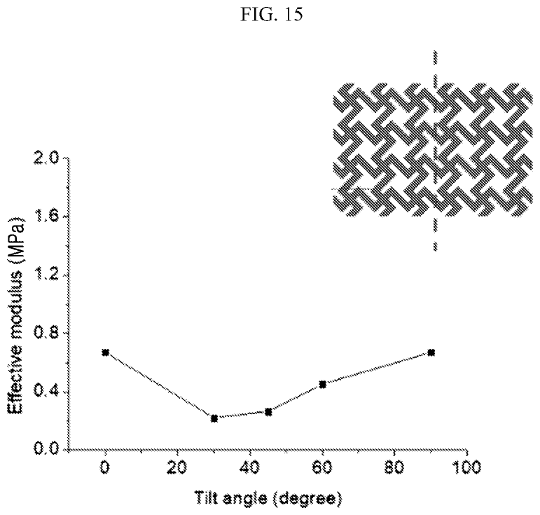

FIG. 14 is a top plan view illustrating an electronic skin sensor patch according to still yet another exemplary embodiment, and FIG. 15 is a graph representing a change in effective modulus according to a tilt angle with respect to a reference line of a stretching direction of a mechanical metamaterial applied to the electronic skin sensor patch illustrated in FIG. 14.

Referring to FIG. 14, an electronic skin sensor patch 50 according to the present exemplary embodiment may be formed of a skin sensor patch for measuring electrocardiogram (ECG). The electronic skin sensor patch 50 includes a patch body 510 including a frame 501 made of a mechanical metamaterial, and sensing units 130, a sensor system unit 140, and a wiring 145 which are fixed to the patch body 510 and measure bio signals.

In the present exemplary embodiment, the mechanical metamaterial of the frame 501 configuring the patch body 510 includes a first frame part 501h extending in a horizontal zigzag direction and a second frame part 501v extending in a vertical zigzag direction, and the first frame part 501h and the second frame part 501v may cross each other to form a crossing point. In this case, an opening 503 surrounded by the first frame part 501h and the second frame part 501v, which connect the adjacent crossing points in the horizontal and vertical directions, may be formed in the patch body 510.

The opening 503 formed in the patch body 510 may include a center opening part 503c and a plurality of branch opening parts 503d integrally extending in directions shifted from the center opening part 503c. The center opening part 503c may have an approximately quadrangular shape, and in this case, the branch opening part 503d may include four branch opening parts 503d extending from the sides of the quadrangular center opening part 503c, respectively.

In the present exemplary embodiment, the first frame part 501h and the second frame part 501v may configure the mechanical metamaterial frame 501 in a Logenz-serpentine structure having a round crest and valley, respectively.

In the present exemplary embodiment, the sensing unit 130, the sensor system unit 140, and the wiring 145 may have the same configurations and characteristics as those of the exemplary embodiment described with reference to FIGS. 2 and 3, so that the repeated descriptions will be omitted.

Referring to FIG. 15, it can be seen that when effective elastic modulus of the electronic skin sensor patch 50 according to the present exemplary embodiment, is calculated while the tensile direction with respect to the reference line is changed, a generally uniform effective elastic modulus in the range of 0.2 to 0.6 MPa is maintained within the tilt angle of 0° to 90°. That is, it can be seen that the change in the elastic modulus according to the tensile direction is not large. However, in the present structure, the change in the elastic modulus according to the direction is larger than that in the Kagome structure, so that it is possible to set an appropriate patch attachment direction according to the skin tensile direction.

The electronic skin sensor patches according to the exemplary embodiments described with reference to FIGS. 10 to 15 have been described based on the structure of the ECG sensor as an example, but the electronic skin sensor patch may be configured by applying the different type of skin sensor, such as a body fluid sensor, to the patch body including the mechanical metamaterial frame having the same structure, which also belongs to the scope of the present invention.

While this invention has been described in connection with what is presently considered to be practical exemplary embodiments, it is to be understood that the invention is not limited to the disclosed embodiments. On the contrary, it is intended to cover various modifications and equivalent arrangements included within the spirit and scope of the appended claims.

### DESCRIPTION OF SYMBOLS

10, 20, 30, 40, 50: Electronic skin sensor patch

101, 201, 301, 401, 501: Mechanical metamaterial frame

110, 210, 310, 410, 510: Patch body

130, 230: Sensing unit

140, 240: Sensor system unit

145, 245: Wiring

114, 214: Sensor system unit cover

115, 215: Wiring cover

What is claimed is:

1. An electronic skin sensor patch which is attached to a skin of a user and measures a bio signal, the electronic skin sensor patch comprising:
a patch body including a frame which is formed with an opening and is made of a mechanical metamaterial;
a sensing unit disposed on a first region of the patch body;
a sensor system unit disposed on a second region of the patch body and configured to maintain a nonadherent state with the skin; and
a wiring disposed along the frame of the patch body and configured to connect the sensing unit and the sensor system unit.

2. The electronic skin sensor patch of claim 1, further comprising:
a sensor system unit cover coupled to the frame of the patch body with the sensor system unit interposed therebetween in the second region of the patch body.

3. The electronic skin sensor patch of claim 2, wherein:
the sensor system unit cover includes a nonadherent surface on a surface facing the skin.

4. The electronic skin sensor patch of claim 1, further comprising:
a wiring cover coupled to the frame of the patch body with the wiring interposed therebetween.

5. The electronic skin sensor patch of claim 1, wherein:
the sensing unit is configured to have one exposed surface to be in contact with the skin.

6. The electronic skin sensor patch of claim 1, wherein:
the sensing unit includes an electrocardiogram (ECG) electrode or an electromyogram (EMG) electrode.

7. The electronic skin sensor patch of claim 6, further comprising:
a spacer interposed between the ECG electrode or the EMG electrode and the frame.

8. The electronic skin sensor patch of claim 1, wherein:
the sensing unit includes a body fluid sensor configured to collect and detect a body fluid.

9. The electronic skin sensor patch of claim 8, wherein:
the body fluid sensor includes:
an opening formed layer including a body fluid passage configured to discharge the body fluid; and
an electrode layer positioned in the opening formed layer and configured to detect a current flowing through the body fluid collected to the body fluid passage.

10. The electronic skin sensor patch of claim 1, wherein:
the frame of the mechanical metamaterial includes a plurality of basic displacement unit bodies, and
in each of the plurality of basic displacement unit bodies,
m polygonal basic unit cells are positioned while being adjacent to each other,
m isolation parts are formed between the m basic unit cells, and
a junction part which connects the basic unit cells to each other is formed between the basic unit cells, and the junction part includes a junction part pattern in which an outer junction part positioned at an outer edge of the basic unit cell and an inner junction part that is not in contact with the outer edge of the basic unit cell are sequentially repeated, and
m is an integer of 4 or 6.

11. The electronic skin sensor patch of claim 10, wherein:
the basic displacement unit body includes a first opening having a variable size, of which an initial unfolding angle is larger than 0° and is equal to or smaller than 15°, at a center.

12. The electronic skin sensor patch of claim 11, wherein:
the first opening has a 3-pointed star shape.

13. The electronic skin sensor patch of claim 11, wherein:
the basic unit cell includes a second opening at a center.

14. The electronic skin sensor patch of claim 13, wherein:
the second opening has a triangular shape.

15. The electronic skin sensor patch of claim 13, wherein:
the six second openings are disposed around each first opening.

16. The electronic skin sensor patch of claim 1, wherein:
the frame made of the mechanical metamaterial includes a first frame part extending in a horizontal zigzag direction and a second frame part extending in a vertical zigzag direction, and
the first frame part and the second frame part cross each other to form a crossing point, and an opening surrounded by the first frame part and the second frame part which connects the adjacent crossing points in horizontal and vertical directions is included.

17. The electronic skin sensor patch of claim 16, wherein:
the opening includes a center opening part and a plurality of branch opening parts integrally extending in directions shifted from the center opening part.

18. The electronic skin sensor patch of claim 17, wherein:
the center opening part has a quadrangular shape, and
the branch opening part includes four branch opening parts extending from sides of the center opening part, respectively.

19. The electronic skin sensor patch of claim 16, wherein:
each of the first frame part and the second frame part includes an angled crest and valley.

20. The electronic skin sensor patch of claim 16, wherein:
each of the first frame part and the second frame part includes a round crest and valley.
